(19) United States
(12) Patent Application Publication
(10) Pub. No.: US 2008/0213124 A1
(43) Pub. Date: Sep. 4, 2008

(54) Device and Method For Waste Breakdown and Disinfection or Treatment

(75) Inventors: Emmanuel Hengl, Issus (FR); Patrick Hengl, Issus (FR)

Correspondence Address: YOUNG & THOMPSON, 209 Madison Street, Suite 500, ALEXANDRIA, VA, 22314, US

(73) Assignee: T.E.M. Technologies Environnement et Medical, Auterive (FR)

(21) Appl. No.: 11/884,124

(22) PCT Filed: Jul. 15, 2003
(86) PCT No.: PCT/FR03/02223

§ 371 Date: Apr. 2, 2008

(30) Foreign Application Priority Data
Jul. 17, 2002 (FR) 02/09008

(51) Int. Cl. A61L 11/00; B02C 18/08

(52) U.S. Cl. 422/1; 422/28; 422/40; 422/291; 422/301; 422/309; 241/82.4; 422/184.1

(57) ABSTRACT

The invention concerns a device mounted on a framework comprising an upper compartment (2) feeding waste (1), equipped with a hopper (6), an intermediate shredding compartment (3), including cutting tools (5), and a lower compartment (4) for disinfecting and evacuating residues. The invention is characterized in that the upper compartment (2) comprises means for conveying waste (1) towards the intermediate compartment (3), means for confining the internal volume of the hopper (6) between the intake orifice (14) comprised therein and its discharge orifice (15) towards the intermediate compartment (3), each of said orifices being equipped with respective closure means; the intermediate compartment (3) emerges directly towards the lower compartment (4), by being in permanent communication, through a passage, with said lower compartment (4), and includes second means for disinfecting the cutting tools (5); and the lower compartment (4) includes a vessel (11) for receiving shredded waste (1′) from the intermediate compartment (3), said vessel (11) being equipped with third disinfecting means for treating the shredded waste (1′) and means for evacuating said waste (1).

The present invention relates to the field of waste treatment by shredding. It more particularly concerns a device and a method for the breakdown and disinfection or treatment of infectious medical waste, but is also applicable to the treatment of putrescible waste such as food waste.

A technique for treating medical waste is known which consists of shredding said waste to reduce its volume and decontaminating said waste so as to eliminate harmful and infectious agents.

For example, patent FR 2 793 143 (HENGL) describes a device for shredding and disinfecting medical waste, which comprises, in successive stages, a feeding compartment, a shredding compartment and a compartment for disinfecting and evacuating residue.

The feeding compartment comprises a hopper, the interior of which is provided with a rolling duct designed to receive the waste, which is conveyed towards the shredding compartment during unwinding of the duct by drive rollers.

A first drawback of this device resides in the opening of the waste reception capacity formed by the duct to the outside. This drawback is especially harmful given that the feeding compartment is in communication with the shredding compartment and, as a result, enables inopportune direct communication of the waste with the outside during its breakdown.

A second drawback is related to the loss of waste reception capacity, due to the reception of said waste inside the duct held taut in the middle of the internal volume of the hopper. Moreover, the consumable nature of the duct generates increased operating costs for the device, which are even more significant in light of the aforementioned loss of volume.

The shredding means equipping the shredding compartment are in particular of the rotary cutting tool type. One problem to be resolved resides in disinfecting these cutting tools, in particular at least between each operating cycle of the device.

For this purpose, the shredding compartment is equipped with disinfection means comprising nozzles for projecting steam inside the shredding compartment, axially along the drive shaft of the rotary cutting tools.

A first drawback resides in the arrangement of the means conveying the steam from the generator to the projection nozzles, which tends to lead to obstruction of said nozzles.

A second drawback resides in unsatisfactory decontamination of the cutting tools.

A third drawback resides in the general organization of the shredding compartment, which makes its maintenance delicate, even more so given that separating means are intended to be placed between the shredding compartment and the disinfection compartment to prevent the latter from coming into communication with the outside.

The waste disinfection compartment comprises an autoclave for receiving the shredded waste. This autoclave is provided with heating means for decontaminating waste and a driving plate to push the decontaminated waste toward a discharge door.

A first drawback resides in insufficient disinfection of the waste, due to its treatment by heat alone and its compacting by the driving plate.

One aim of the present invention is to propose a shredding and decontamination device for infectious medical waste, arranged so as to resolve the aforementioned drawbacks. More particularly, the purpose of the present invention is to propose a device of this type which is organized so as to allow reliable and effective waste treatment in a secure environment.

The device according to the present invention is, generally, a device for waste breakdown and disinfection or treatment, more particularly adapted to infectious medical waste but which can be applied to all other types of waste requiring similar treatment.

This device is, in a known manner, mounted on a framework and comprises a successive series of compartments, including an upper compartment feeding waste, equipped with a hopper, an intermediate shredding compartment, including cutting tools to break down the waste, and a lower compartment for disinfecting and evacuating residues.

According to the invention,
the upper compartment comprises means for conveying waste towards the intermediate shredding compartment, means for confining the internal volume of the hopper between the intake orifice comprised therein and its discharge orifice towards the intermediate compartment, each of these orifices being equipped with respective closure means, able to be maneuvered between a disengaged position and a closed position of the corresponding orifice, and first means for disinfecting the waste and the hopper;
the intermediate compartment emerges directly towards the lower compartment, by being in permanent communication, through a passage, with said lower compartment, and includes second means for disinfecting the cutting tools, and
the lower compartment includes a vessel for receiving shredded waste from the intermediate compartment, said vessel being equipped with third disinfecting means for treating the shredded waste and means for evacuating treated waste through an evacuation door.

These arrangements are such that, for a given waste treatment cycle, the upper compartment can be opened to the external environment in a first step for the introduction of waste, then isolated by closing the intake orifice of the hopper. The waste is then conveyed towards the intermediate compartment, then, in a second step, the waste is continuously shredded and introduced inside the vessel, up to a desired quantity corresponding to that of the waste previously introduced into the hopper. Lastly, in a third step, the upper compartment is isolated by closing the discharge orifice of the hopper, for simultaneous operations to disinfect the tools and the shredded waste contained in the vessel.

One will also understand that each of the compartments of the device is equipped with its own disinfecting means, such that the waste, during its progression inside the device, can be disinfected in each of the compartments. Moreover, each compartment, thanks to its own disinfecting means, can be disinfected after each treatment cycle. Likewise, the upper compartment, before maneuvering of its upper closure means toward the disengaged position, can be disinfected. Thus, before any operation by personnel at the upper compartment, the internal surfaces of this compartment will be disinfected, which makes it possible to reduce the risk of contamination through contact.

One will also notes that the device according to the invention is made up of three impermeable compartments which can be disinfected independently of each other before opening toward the external environment and especially prior to any disassembly and/or any operation by the personnel responsible for the maintenance or operation of the device.

According to one preferred embodiment of the invention, the closing means of the intake orifice of the hopper comprise a cover articulated to said hopper.

According to one preferred embodiment of the invention, the closure means of the discharge orifice of the hopper comprise an isolation valve arranged between the hopper and the intermediate compartment, comprising a valve body integral with the hopper on one hand and the intermediate compartment on the other, said valve body comprising a through opening for the passage of waste and said valve also comprising a slide valve slidingly mounted in the valve body between a closed position, in which the through opening is closed and the hopper and intermediate compartment are isolated from each other, and a disengaged position, in which the through opening is disengaged and communication between the hopper and the intermediate compartment is established.

According to another characteristic of the invention, the valve body, around the through opening, is equipped with an inflatable peripheral seal, for sealing between the upper compartment and the intermediate compartment, by elastic compression of the slide valve sliding against the framework through an inflatable seal, and to facilitate sliding of the slide valve by freeing it from the seal, pressure-free.

The inflatable nature of this seal makes it possible to obtain quality sealing between the upper compartment and the intermediate compartment, through elastic compression of the slide valve against the valve body using an inflatable seal. Moreover, the inflatable nature of the seal allows a reduction of its volume when it is inflated prior to opening of the discharge orifice of the hopper, to facilitate sliding of the slide valve by disengaging the seal pressure-free.

According to another aspect of the present invention, and given the access to the internal volume of the hopper during the shredding operation, the means for conveying waste from the upper compartment towards the intermediate compartment are made up of a driving member in the form of a rod going straight through said cover, articulated omni-directionally to the cover on one hand and having a limited axial movable range on the other hand, so as to allow it access to the entirety of the internal volume of the hopper and to the internal surfaces thereof, said member being sealably connected to said cover.

This omni-directional articulation and this axial movable range of the driving member enable it access to the entire internal volume of the hopper and its internal surfaces, thanks to which the driving member is able to direct all of the waste towards the intermediate compartment, including waste adhering to the internal surface of the hopper.

The driving member may be connected to the cover by a bellows or an impermeable articulation sphere; it may also be slidingly engaged in a cylindrical part connected to a flexible disk, itself connected to said cover, this cylindrical part defining an internal chamber, crossed by the driving member, filled with a bactericidal product.

According to another aspect of the present invention, the upper compartment is equipped with means for admitting a treatment fluid (liquid, gas or vapor) comprising at least any one of first means for admitting a disinfecting fluid for treatment of the hopper (liquid, gas or vapor) and second means for admitting a disinfecting liquid for bath treatment of the waste.

One will note that bath treatment of the waste in the upper compartment is made possible thanks to the presence of the means for confining the hopper, which advantageously constitute means for holding the disinfection liquid for waste treatment by immersion.

The first means for admitting a disinfecting fluid (liquid, gas or vapor) for treatment of the hopper are, for example, made up of at least one projection nozzle for a corresponding fluid (liquid, gas or vapor) which emerges through the cover inside the volume of the hopper.

The second means for admitting a disinfecting liquid, such as sodium hydroxide, for bath treatment of the waste, are produced simply by exploiting the possibility of accessing the internal volume of the hopper through its intake orifice, when the cover is open and the discharge orifice of the hopper is closed, in particular by the slide valve.

The cover is preferably formed in a dome, to promote the flow of the disinfecting fluid of the hopper on its internal surface, with a view to its draining towards the internal volume of the hopper.

According to another aspect of the present invention, the cutting tools are confined to the inside of a frame arranged in a framework of lateral walls, which is removably mounted on the framework like a slide valve. This arrangement and mounting of the frame are such that they allow easy placement and, inversely, removal of the tools, with a view to their maintenance and/or that of the device.

The interest of the second disinfecting means is to enable cleaning and disinfection of the tools prior to their removal and handling by the user, in the case, for instance, of a malfunction of the device during an operating cycle involving its interruption.

According to another characteristic of the invention, at least the second and third disinfection means comprise a shared hot steam generator and each have a circuit for injecting this hot steam into their internal volumes.

This arrangement allows disinfection of these compartments at least through the action of the heat. The interest of this characteristic is to achieve a suitable temperature, before treatment, in the intermediate compartment and possibly the lower compartment, and to bring the cutting tools of the intermediate compartment to a suitable temperature.

Preferably, hot water steam is used to disinfect the intermediate compartment and the lower compartment, this hot steam possibly being able to be supplemented by at least one bactericidal agent to reinforce the treatment action. It does, however, go without saying that any gaseous fluid or liquid having bactericidal properties may be used.

According to another characteristic of the invention, the cutting tools are organized in two parallel series of cutting tools and the tools of each series are mounted on a support shaft for rotational driving unique to the series of tools, said tools being mounted on said shafts so as to be fixed in rotation and translation relative to the latter. Moreover, the tools of each series are arranged regularly spaced from each other such that between two consecutive tools there is an interval, the width of which is greater than the thickness of each tool, and the two series of tools are arranged such that they are intertwined in the sense that the tools from each series penetrate the intervals between the tools from the other series. The two drive shafts are driven in rotation in opposite directions relative to each other and at different rotational speeds.

According to another aspect of the invention, the cutting tools of each series are separated from each other by spacers engaged on the corresponding shaft.

According to another aspect of the present invention, the second disinfection means comprise at least one channel for conveying a disinfecting fluid (liquid, gas or vapor). This channel is arranged through at least one of the side walls of the frame following a direction radial to the cutting tools and extends to emerge in the intervals between the tools, such that the disinfecting liquid can be directly brought from the outside of the frame and projected against the lateral surfaces of the cutting tools along a projection direction contained in a plane parallel to these surfaces. One will understand that are preferably multiple channels present in a number corresponding to that of the intervals to which they are each allocated.

According to another aspect of the present invention, the third disinfection means equipping the vessel of the lower compartment comprise, alone or, preferably, in combination:

a) means for evacuating the internal volume of the vessel. One will note that due to the permanent communication between the lower compartment and the intermediate compartment, the latter is evacuated simultaneously with the evacuation of the vessel.

b) means for pressurized admission of a disinfecting fluid inside this vessel, such as water vapor additionally mixed with a treating agent, but it will be possible to use a disinfecting liquid or gas.

c) means for mixing the shredded waste, to mix it during the disinfection phase.

These mixing means are advantageously made up of a worm able to be alternatingly maneuvered in two rotational directions, to allow one to adapt the mixing operation time according to the delivery of shredded waste from the intermediate compartment. Moreover, said worm is advantageously exploited to form the means for evacuating the shredded waste so as to convey it towards the evacuation door, from a continuous rotation of the worm in the corresponding direction.

One will note that the means for admitting a disinfecting fluid (gas or liquid or vapor) inside the vessel are advantageously made up of nozzles arranged in the rotating shaft of the worm and occupying a radial position relative to said shaft, said nozzles being supplied with fluid from the outside, through a channel extending axially inside the rotational shaft of the worm.

Moreover, drain grid for the treated waste is advantageously arranged at the outlet of the vessel, and the device also comprises means for evacuating exudates, arranged at the base of the vessel.

Other aims, advantages and characteristics of the present invention will appear upon reading the description of one preferred embodiment provided as a non-limiting example, in reference to the appended figures, in which:

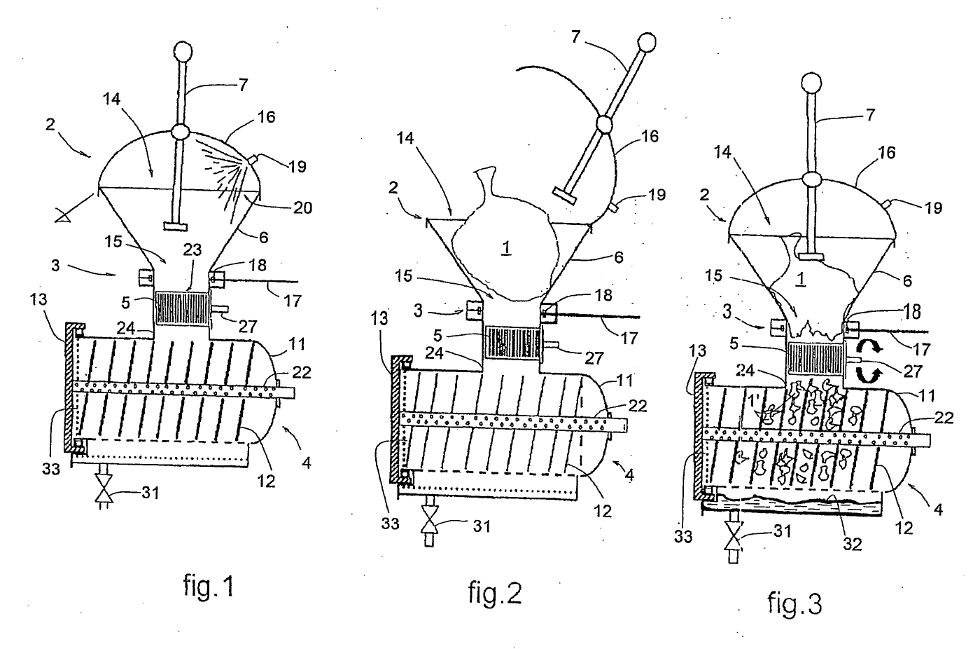

FIGS. 1 to 10 diagrammatically illustrate one embodiment of the device according to the invention, at various successive stages of its implementation;

FIG. 11 is a diagrammatic cross-section of the device illustrated in the preceding figures;

FIG. 12 is a diagrammatic cross-section of a frame supporting the cutting tools equipping a device illustrated in the preceding figures;

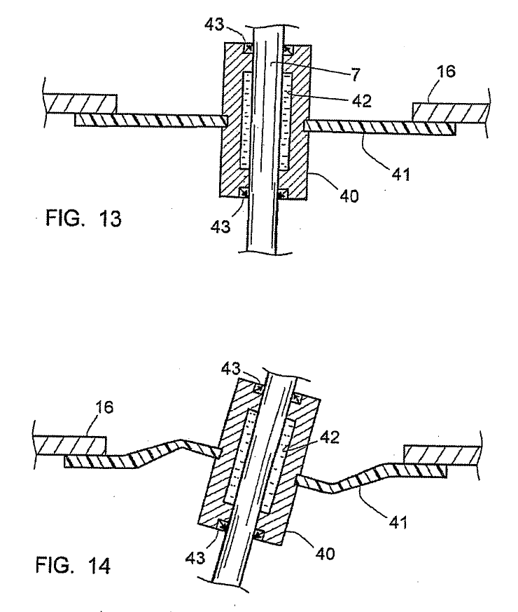

FIGS. 13 and 14 are cross-sections of a detail, according to another embodiment.

In FIGS. 1 to 11, a device according to the present invention is illustrated, primarily comprising three compartments 2, 3, 4 in a stepwise succession, including one upper compartment 2 feeding waste, an intermediate compartment 3 shredding waste and a lower compartment 4 treating the shredded waste.

The upper compartment 2 supplying waste 1 is made up primarily of a hopper 6 topped by a cover 16. The lower orifice of the hopper 6 can be closed by an isolation valve arranged between the hopper 6 and the intermediate compartment 3, comprising a valve body integral with the hopper on one hand and the intermediate compartment on the other hand, said valve body comprising a through opening for the passage of waste. Said valve also comprises a slide valve 17 slidingly mounted in the valve body between a closed position, in which the through opening is closed and the hopper and the intermediate compartment are isolated from each other, and a disengaged position, in which the through opening is disengaged and communication between the hopper and the intermediate compartment is established.

The valve body around the through opening is equipped with an inflatable peripheral seal 18, for sealing between the upper compartment 2 and the intermediate compartment 3, by elastic compression of the slide valve 17 against the framework 24 through the inflatable seal 18, and to facilitate sliding of the slide valve 17 by freeing it from the seal 18, without pressure.

A driving member 7, in the form of a rod, goes straight through the cover 16 and is articulated omni-directionally and sealably to said cover. This articulation is produced through a sphere receiving the sliding member 7, mounted pivotably in a seat integral with the cover. This driving member 7 has, through its sliding relative to said sphere, a limited axial movable range, in one direction and the other, such that its end inside the hopper 6 can access the entire internal volume of said hopper 6 and the internal surfaces thereof.

A nozzle 19 for projecting disinfecting fluid 20 (liquid, gas or vapor) in the hopper 6 emerges through the cover 16. One will note that the latter is formed in a dome and is advantageously transparent to allow visual access to the waste 1 when it is conveyed towards the intermediate compartment 3.

The intermediate compartment 3 is reserved for shredding waste 1 using rotary cutting tools 5 housed inside a removable frame 23. These rotary cutting tools 5 are driven in rotation from the outside of the intermediate compartment 3 by two shafts 27 which support them, and which emerge outside the frame 23.

In FIG. 11, the intermediate compartment 3 is primarily made up of a frame 23 removably supported by the framework of the device. More particularly, this frame 23 is organized in a removable drawer containing the rotary cutting tools 5, which are distributed in two pluralities of cutting tools 5 intertwined on respective rotating shafts 27, which are driven from the outside of the frame 23. One will note that the frame 23, formed in a framework, has a passage 8 to its lower part for the disinfecting liquid 21 from the lower compartment 4, and a passage 9 to its upper part, for the introduction of waste 1.

The shafts 27 are guided in rotation by bearings integral with the frame 23 of the intermediate compartment 3.

The cutting tools 5 each comprise two or several radial blades and are each provided with a polygonal through bore through which they are engaged on their respective shaft 27, said shaft having a polygonal straight cross-section to ensure, in combination with the polygonal shape of the bore of each cutting tool, the rotational connection of each cutting tool on said shaft.

The cutting tools 5 are maintained constantly separated from each other by spacers 26 engaged on their respective shafts 27, the thickness of each spacer 26 being greater than the thickness of each cutting tool 5, such that the cutting tools supported by one shaft are able to penetrate the intervals between the cutting tools supported by the other shaft. Two series of cutting tools are thus formed, in mutual interpenetration.

According to the preferred embodiment of the invention, the thickness of the spacers 26 is greater by several tenths of millimeters than that of the cutting tools.

Thus, the gap between the cutting tools 5 of one series and the cutting tools 5 of the other series, measured between one of the flat surfaces of a tool from one series and the contiguous flat surface of a tool from the other series, is very small and not sufficient to allow the passage of small objects such as pins, needles and others. Moreover, the circular geometrical envelope associated with each cutting tool 5 (circular trajectory having a larger diameter traveled by the end of each tooth of the cutting tools) is separated from the cylindrical surface of the opposite spacer 26 located on the other shaft, by several tenths of mm, so as to be able to grasp and sever small objects such as needles and others. The two shafts are driven in rotation in opposite directions relative to each other such that the waste is grasped in the upper part and discharged in the lower part. Advantageously, they are driven according to different rotation speeds so as to add a stretching effect to the cutting effect, which improves the breaking down of this waste.

In FIG. 12, it is shown that one of the side walls of the frame 23 comprises the distribution circuit for a disinfecting fluid formed by channels 28 for conveying said fluid from the outside of the device, going through said wall along a direction radial to the cutting tools, towards the lateral surfaces of the cutting tools 5 along a projection direction contained in a plane parallel to these surfaces.

Preferably, each side wall comprises a side element 29 parallel to the drive shafts 27 with which a stack of wall elements 30a, 30b is associated, each wall element 30a, 30b extending perpendicularly to the drive shafts 27.

Preferably, as shown in FIG. 12, two types of wall elements 30a, 30b are provided on the two opposite lateral walls parallel to the shafts 27, these elements 30a, 30b being arranged alternating. The elements 30a of the first type penetrate the intervals between the cutting tools 5 and each comprise a recess in a circular arc of circumference to fit the spacers 26 and to come closer to the latter, and the elements 30b of the second type also each have a recess in a circular arc of circumference and are brought by these recesses as close as possible to the trajectory of the end of the teeth of the cutting tools 5.

Preferably, each element 30a, 30b has the shape of a stud whereby it engages in a slot shape formed in the side element 29.

The circuit for distributing the treatment fluid is formed by a first longitudinal channel formed in the studs and slots and by transverse channels 28 hollowed in the lateral surfaces of the wall elements 30a, 30b, radially to the drive shafts 27.

These channels 28 are formed by grooves and said grooves are found opposite the flat surfaces of the cutting tools 5.

The lower compartment 4 for treating shredded waste 1′ comprises a vessel 11 for receiving said waste, a worm 12 for mixing it and conveying it towards an evacuation door 13, and a vacuum pump connected to the volume defined by the vessel 11, for evacuating this volume.

The worm 12 comprises a hollow shaft having radial holes 22 and is connected to a vapor production source. It thus enables the introduction of the vapor inside the vessel 11, which constitutes a disinfecting fluid for the shredded waste 1″, and forms mixing means for said shredded waste 1″, to mix it during the disinfection phase. This worm 12 also forms means for evacuating said shredded waste 1″ towards the door 13.

One will note the advantageous presence of a drain grid 33 placed directly upstream from this evacuation door 13. One will also note the presence of means 31 for evacuating exudates 32, these means comprising an outlet gate in particular.

After treatment, the waste can be compacted in the lower compartment through the pressing action exerted by the worm 12 on said waste; the door being closed, the waste is therefore compressed between the worm 12 and the door 13 in order to reduce its volume before evacuation.

FIGS. 1 to 10 successively illustrate the steps for implementing a waste treatment cycle, comprising:

FIG. 1: disinfection of the upper compartment 2 by projecting the disinfecting liquid 20, before opening the cover 16 closing the intake orifice 14 of the hopper 6 for waste 1;

FIG. 2: opening the cover 16 and introduction of waste 1 in the hopper 6, then closing of the cover 16;

FIG. 3: conveying waste through a driving member 7 from the hopper 6 towards the cutting tools 5 for its shredding, and introduction of shredded waste 1′ inside the vessel 11;

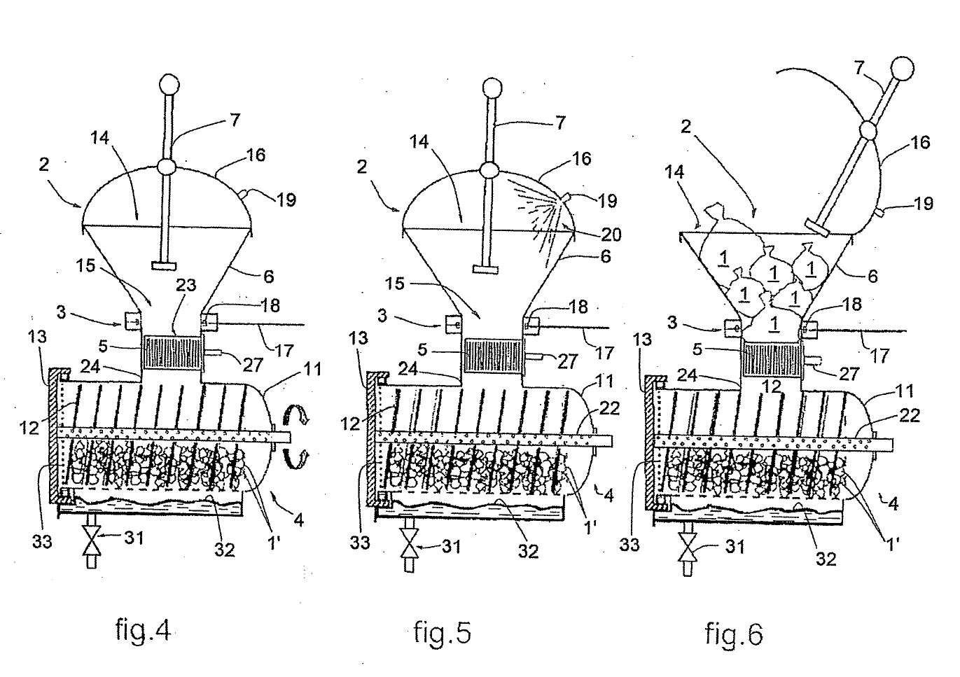

FIG. 4: mixing of the shredded waste 1′ inside the vessel 11, from a rotation of the worm 12 alternating in both directions;

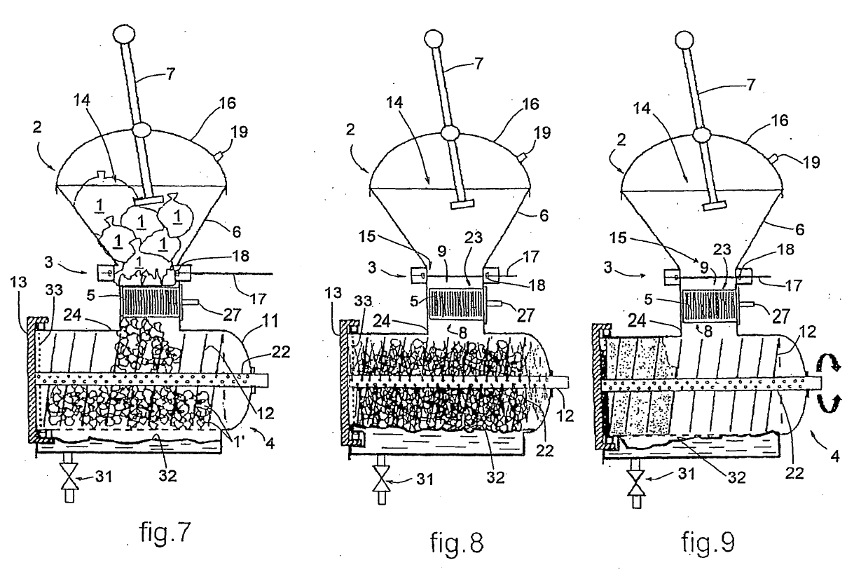

FIGS. 5, 6 and 7: in the case of introduction inside the hopper 6 of a new quantity of waste 1, any disinfection of the upper compartment 2 by projecting a disinfecting liquid 20 (FIG. 5), then introduction of waste 1 (FIG. 6) and driving of this waste 1 towards the intermediate compartment 3 (FIG. 7);

FIG. 8: closing of the sliding valve 17 to isolate the hopper 6 and the intermediate 3 and lower 4 compartments, mixing of the shredded waste 1′, evacuation of the internal volume of the vessel 11 and projection of disinfecting liquid 20, for disinfecting of the shredded waste 1′ and cutting tools 5, by circulation through a communication passage 8, arranged between the intermediate 3 and lower 4 compartments;

FIG. 9: venting of the internal volume of the vessel 11, draining against the grid 33 and conveying of the treated waste 1″ towards the evacuation door 13, then emptying of exudates 32 through rotation of the worm 12 in the corresponding direction;

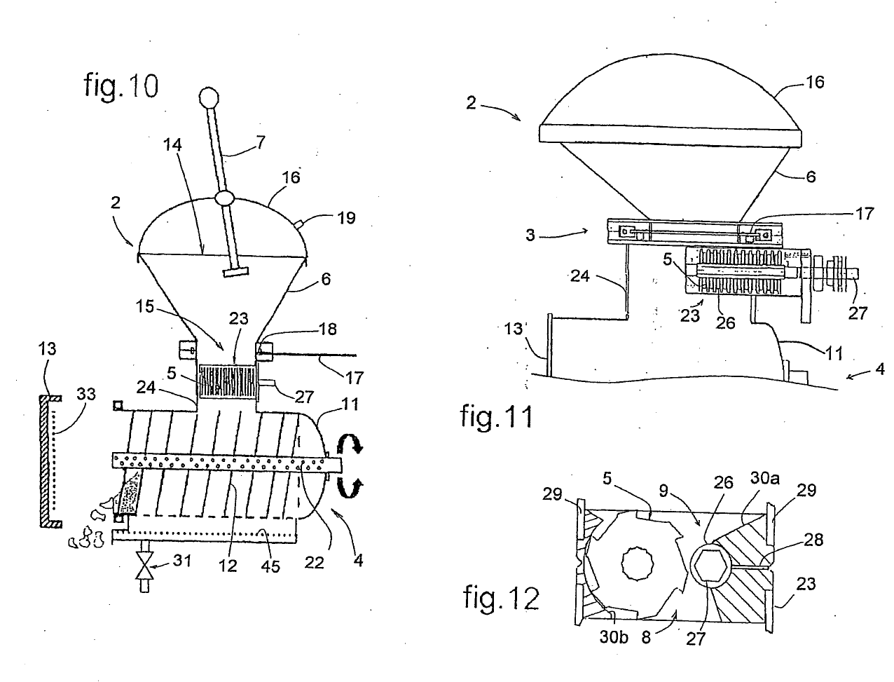

FIG. 10: opening of the door 13 and evacuation of the treated waste 1″ outside the vessel 11.

FIGS. 13 and 14 illustrate another embodiment of the articulation of the driving member 7 to the cover 16. In this case, the member 7 is slidingly engaged in a cylindrical part 40 connected to a disk 41 of a silicone elastomer, itself connected to the cover 16, by sticking. Said cylindrical part 40 defines an internal chamber 42, passed through by the driving member 7, filled with a bactericidal gel and is closed at its ends by lip seals 43.

One will note that the successive implementations of the various means of the device according to the invention can advantageously be controlled from a machine.

It goes without saying that the present invention can receive any arrangements and variations from the field of technical equivalents without going outside the scope of the present patent. Thus, injecting a disinfecting fluid (vapor) in the vessel 11 may be done using means other than holes 22, in particular at the bottom part of this vessel 11, through a grid 45 filtering the exudates 32 which may be contained in the vessel 11 (cf. FIG. 10).

1- Device for the breakdown and disinfection or treatment of waste, in particular of infectious medical waste (1), mounted on a framework (24) and comprising a successive series of compartments (2, 3, 4), including an upper compartment feeding waste (2), equipped with a hopper (6), an intermediate shredding compartment (3), including cutting tools (5) to break down the waste, and a lower compartment (4) for disinfecting and evacuating residues;
device characterized in that:
the upper compartment comprises means (7) for conveying waste towards the intermediate shredding compartment (3), means for confining the internal volume of the hopper (6) between the intake orifice (14) comprised therein and its discharge orifice (15) towards the intermediate compartment (3), each of these orifices (14, 15) being equipped with respective closure means (16, 17), able to be maneuvered between a disengaged position and a closed position of the corresponding orifice, and first means (19) for disinfecting the waste (1) and the hopper (6);
the intermediate compartment (3) emerges directly towards the lower compartment (4), by being in permanent communication, through a passage (8), with said lower compartment (4), and includes second means for disinfecting the cutting tools (5), and
the lower compartment (4) includes a vessel (11) for receiving shredded waste from the intermediate compartment (3), said vessel (11) being equipped with third disinfecting means (21, 22) for treating the shredded waste and means (12) for evacuating treated waste through an evacuation door (13).

2- Device according to claim 1, characterized in that the closing means of the intake orifice (14) of the hopper (6) comprise a cover (16) articulated to said hopper (6).

3- Device according to claim 1 or claim 2, characterized in that the closure means of the discharge orifice (15) of the hopper (6) comprise an isolation valve arranged between the hopper (6) and the intermediate compartment (3), comprising a valve body integral with the hopper (6) on one hand and the intermediate compartment (3) on the other, said valve body comprising a through opening for the passage of waste and said valve also comprising a slide valve (17) slidingly mounted in the valve body between a closed position, in which the through opening is closed and the hopper (6) and intermediate compartment (3) are isolated from each other, and a disengaged position, in which the through opening is disengaged and communication between the hopper (6) and the intermediate compartment (3) is established.

4- Device according to claim 3, characterized in that the valve body, around the through opening, is equipped with an inflatable peripheral seal (18), for sealing between the upper compartment (2) and the intermediate compartment (3), by elastic compression of the slide valve (17) sliding against the framework (24) through an inflatable seal (18), and to facilitate sliding of the slide valve (17) by freeing it from the seal (18), pressure-free.

5- Device according to anyone of claims 2 to 4, characterized in that the means for conveying waste from the upper compartment (2) towards the intermediate compartment (3) are made up of a driving member (7) in the form of a rod going straight through said cover (16), articulated omni-directionally to the cover (16) on one hand and having a limited axial movable range on the other hand, so as to allow it access to the entirety of the internal volume of the hopper (6) and to the internal surfaces thereof, said member being sealably connected to said cover (6).

6- Device according to claim 5, characterized in that the driving member (7) is slidingly engaged in a cylindrical part (40) connected to a flexible disk (41), itself connected to said cover (16), this cylindrical part (40) defining an internal chamber (42), crossed by the driving member (7), filled with a bactericidal product.

7- Device according to anyone of claims 1 to 6, characterized in that the upper compartment (2) is equipped with means for admitting a treatment fluid, such as a liquid, gas or vapor, comprising at least any one of first means (19) for admitting a disinfecting fluid (20) for treatment of the hopper (6), such as a liquid, gas or vapor, and second means for admitting a disinfecting liquid for bath treatment of the waste (1).

8- Device according to claim 7, characterized in that the means (16, 17)) for confining the hopper (6) constitute means for holding the disinfection liquid for waste treatment by immersion.

9- Device according to claims 2 and 7, characterized in that the first means for admitting a disinfecting fluid (20), such as a liquid, gas or vapor, for treatment of the hopper (6) are made up of at least one projection nozzle (19) for a corresponding fluid (liquid, gas or vapor) which emerges through the cover (16) inside the volume of the hopper (6).

10- Device according to anyone of claims 2 to 9, characterized in that the cover (16) is formed in a dome, to promote the flow of the disinfecting fluid (20) of the hopper (6) on its internal surface, with a view to its draining towards the internal volume of the hopper (6).

11- Device according to anyone of claims 1 to 9, characterized in that the cutting tools (5) are confined to the inside of a frame (23) arranged in a framework of lateral walls, which is removably mounted on the framework (24) like a slide valve, to allow easy placement and, inversely, removal, of the tools (5).

12- Device according to anyone of claims 1 to 9, characterized in that the cutting tools (5) are organized in two parallel series of cutting tools (5) and the tools (5) of each series are mounted on a support shaft (27) for rotational driving unique to the series of tools, said tools being mounted on said shafts (27) so as to be fixed in rotation and translation relative to the latter, the tools of each series being arranged regularly spaced from each other such that between two consecutive tools there is an interval, the width of which is greater than the thickness of each tool, and the two series of tools being arranged such that they are intertwined in the sense that the tools from each series penetrate the intervals between the tools from the other series.

13- Device according to claim 12, characterized in that the two drive shafts (27) are driven in rotation from the outside of the framework (24), in opposite directions relative to each other and at different rotational speeds.

14- Device according to claim 12 or claim 13, characterized in that the cutting tools (5) of each series are separated from each other by spacers (26) engaged on the corresponding shaft.

15- Device according to anyone of claims 11 to 14, characterized in that the second disinfection means comprise at least one channel (28) for conveying a disinfecting fluid (liquid, gas or vapor), which is arranged through at least one of the side walls of the frame (23) following a direction radial to the cutting tools (5) and extends to emerge in the intervals between the tools, such that the disinfecting liquid can be directly brought from the outside of the frame (23) and projected against the lateral surfaces of the cutting tools (5) along a projection direction contained in a plane parallel to these surfaces.

16- Device according to anyone of claims 1 to 15, characterized in that the third disinfection means equipping the vessel (11) of the lower compartment (4) comprise means for evacuating the internal volume of the vessel (11).

17- Device according to anyone of claims 1 to 16, characterized in that the third disinfection means (22) equipping the vessel (11) of the lower compartment (4) comprise means for pressurized admission of a disinfecting fluid (21) inside this vessel (11).

18- Device according to anyone of claims 1 to 17, characterized in that the third disinfection means equipping the vessel (11) of the lower compartment (4) comprise means (12) for mixing the shredded waste (1′), to mix it during the disinfection phase.

19- Device according to claim 18, characterized in that the mixing means are made up of a worm (12) able to be alternatingly maneuvered in two rotational directions, to allow one to adapt the mixing operation time according to the delivery of shredded waste from the intermediate compartment (3).

20- Device according to claim 19, characterized in that the means for evacuating the shredded waste (1′) are made up of the worm (12) conveying it towards the evacuation door (13), from a continuous rotation of the worm (12) in the corresponding direction.

21- Device according to anyone of claims 1 to 20, characterized in that it comprises a drain grid (33) for the treated waste (1″) arranged at the outlet of the vessel (11), and means (31) for evacuating exudates (32), arranged at the base of the vessel (1).

22- Method for the treatment of waste by breakdown and disinfection using the device according to claim 1, characterized in that, for a given waste treatment cycle, it includes the following steps:
opening of the upper compartment (2) to the external environment for the introduction of waste (1), then isolated by closing the intake orifice (14) of the hopper (6) and conveying of the waste (1) towards the intermediate compartment (3)
continuous shredding and introduction of the shredded waste (1′) inside the vessel (11), up to a desired quantity corresponding to that of the waste (1) previously introduced into the hopper (6);
isolating the upper compartment (2) by closing the discharge orifice (15) of the hopper (6);
simultaneous operations to disinfect the tools and the shredded waste (1′) contained in the vessel (11).

23- Method according to claim 22, using a device according to the claims 1 to 19, characterized in that it consist in carrying out the steps of a waste treatment cycle, comprising the following steps:
a) disinfection of the upper compartment (2) by projecting the disinfecting liquid, before opening the cover (16) closing the intake orifice (14) of the hopper (6) for waste (1);
b) opening the cover (16) and introduction of waste (1) in the hopper (6), then closing of the cover (16);
c) conveying waste (1) through a driving member from the hopper (6) towards the cutting tools for its shredding, and introduction of shredded waste (1′) inside the vessel (11);
d) mixing of the shredded waste (1′) inside the vessel (11), from a rotation of the worm (12) alternating in both directions;
e) in the case of introduction inside the hopper (6) of a new quantity of waste (1), any disinfection of the upper compartment (2) by projecting a disinfecting liquid, then introduction of waste (1) and driving of this waste (1) towards the intermediate compartment (3);
f) closing of the sliding valve (17) to isolate the hopper (6) and the intermediate (3) and lower (4) compartments, mixing of the shredded waste (1′);
g) evacuation of the internal volume of the vessel (11) and projection of disinfecting liquid, for disinfecting of the shredded waste (1′) and cutting tools (5), by circulation through a communication passage, arranged between the intermediate (3) and lower (4) compartments;
h) venting of the internal volume of the vessel (11), draining against the grid and conveying of the treated waste (1″) towards the evacuation door (13), then emptying of exudates (32) through rotation of the worm (12) in the corresponding direction;
i) opening of the door (13) and evacuation of the treated waste (1″) outside the vessel (11).
